(19) United States
(12) Patent Application Publication
(10) Pub. No.: US 2015/0336857 A1
(43) Pub. Date: Nov. 26, 2015

(54) PROCESS FOR PREPARING ETHYLENE AND/OR PROPYLENE

(71) Applicant: SHELL OIL COMPANY, Houston, TX (US)

(72) Inventors: Leslie Andrew CHEWTER, Amsterdam (NL); Ye-Mon CHEN, Sugar Land, TX (US); Timothy Michael NISBET, Amsterdam (NL); Sivakumar SADASIVAN VIJAYAKUMARI, Gonzales, LA (US); Jeroen VAN WESTRENEN, Amsterdam (NL)

(21) Appl. No.: 14/412,692

(22) PCT Filed: Jul. 1, 2013
(86) PCT No.: PCT/EP2013/063838

§ 371 Date: Jan. 5, 2015

Related U.S. Application Data
(60) Provisional application No. 61/667,662, filed on Jul. 3, 2012.

(51) Int. Cl. C07C 1/24; B01J 8/02

(52) U.S. Cl. CPC C07C 1/24; B01J 8/02; B01J 2208/00902; B01J 2208/00752

(57) ABSTRACT

The present invention provides a process for preparing ethylene and/or propylene, wherein an oxygenate feedstock is contacted with a molecular sieve-comprising catalyst at a temperature in the range of from 350 to 500° C. to obtain a reactor effluent comprising ethylene and/or propylene and the oxygenate feedstock is contacted with the catalyst in a riser reactor having a reactor wall defining a flow trajectory towards a downstream outlet for reactor effluent, wherein at least oxygenate feedstock and catalyst are provided at one or more upstream inlets of the riser reactor and wherein a water quench medium is admitted to the riser reactor at one or more of locations along the length of the flow trajectory through a plurality of inlets distributed along the periphery of the reactor wall. The invention further provides a reaction system suitable for preparing ethylene and propylene.

### FIELD OF THE INVENTION

This invention relates to a process for preparing ethylene and/or propylene and a reaction system suitable therefore.

### BACKGROUND TO THE INVENTION

Conventionally, ethylene and propylene are produced via steam cracking of paraffinic feedstocks including ethane, propane, naphtha and hydrowax. An alternative route to ethylene and propylene is an oxygenate-to-olefin (OTO) process. Interest in OTO processes for producing ethylene and propylene is growing in view of the increasing availability of natural gas. Methane in the natural gas can be converted, for instance, to methanol or dimethylether (DME), both of which are suitable feedstocks for an OTO process.

In an OTO process, an oxygenate such as methanol is provided to a reaction zone comprising a suitable conversion catalyst and converted to ethylene and propylene.

The conversion of oxygenates, such as methanol, to olefins is an exothermic process. Consequently, as the conversion process progresses, the temperature of the reaction mixture in the reactor increases. Such a temperature increase is undesired as it may accelerate deactivation of the catalyst. For instance, U.S. Pat. No. 4,071,573 describes the deactivation of molecular sieve-comprising catalyst s due to the temperature increase in exothermic methanol to gasoline processes.

In US2009/0163756, a method for removing heat from an oxygenate conversion process is described. In the process of US2009/0163756, cooling tubes are disposed within the reactor, extending adjacent to the reactor wall from an upper part of the reactor to a lower part of the reactor. A cooling medium is passed through the cooling tubes to remove the heat of reaction.

A disadvantage of the process of US2009/0163756 is the need to provide cooling tubes within the reactor which complicates the design of the reactor, while the presence of the cooling tubes influences the flow regime in the reactor.

In U.S. Pat. No. 4,071,573, a process is described, wherein the temperature in the conversion reactor is controlled in several ways including addition of a quench medium, to the reaction mixture inside the reactor or cooling of the catalyst prior to entering the reactor. The preferred quench medium in U.S. Pat. No. 4,071,573 is steam, but also water is mentioned. Quench medium is provided to the reaction mixture by one or more distributor grids, which are positioned in the reactor vessel cross-section and extending into the reaction mixture. In addition, a plurality of grid means are provided, positioned in the reactor vessel cross-section and extending into the reaction mixture. The grid means are provided to provide dispersion of the quench medium gas bubbles.

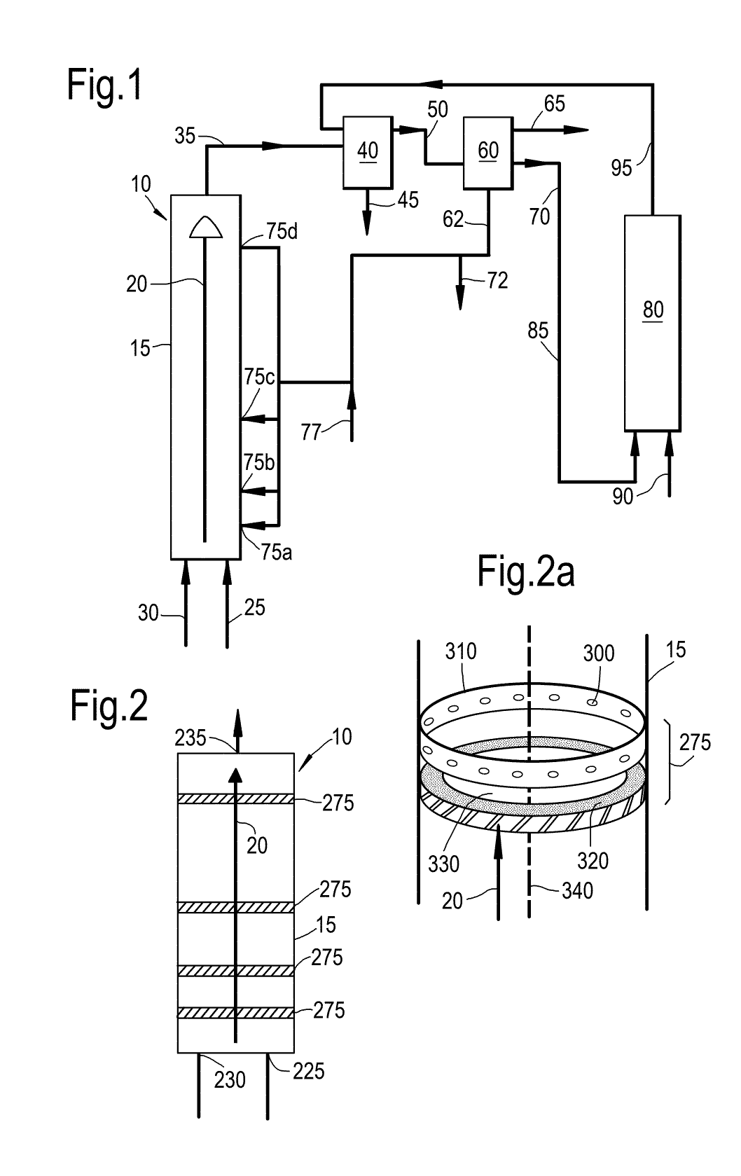

A disadvantage of the process of U.S. Pat. No. 4,071,573 is that it requires providing distributor grids extending into the reaction medium. Due to the high temperature of the reaction medium, when water is passed through the distributor grid the water may be partially vaporized inside the grid generating a two phased water/steam flow. Such a two phased flow may cause flow instability inside the distributor and resultantly a poor distribution of the water/steam in the reaction mixture. Consequently, it is necessary to additionally provide the grid means 6 in between subsequent distributor grids 8 to provide dispersion of the quench medium gas bubbles as shown in FIG. 1 of U.S. Pat. No. 4,071,573. Moreover, these distributors typically cannot ensure the formation of fine droplets, which are preferred for fast quench effects. Distributors as used in U.S. Pat. No. 4,071,573 only use liquid pressure to generate droplets. Even with very smaller outlets distributor, it will generate droplets on the order of several mm size, which are less effective for the desired quench purposes. In addition, the grid means, as used in U.S. Pat. No. 4,071,573, are sensitive damage or even disintegration leading to inefficiency or even interruption of the process.

There is a need in the art for an improved process for producing ethylene and/or propylene from an oxygenate feedstock, wherein the temperature of the reaction mixture, and in particular the catalyst, in the reactor is controlled during an exothermic oxygenate to olefins process by providing water as a quench medium.

### SUMMARY OF THE INVENTION

It has now been found that in an oxygenate to olefins process, the temperature of the reaction mixture, and in particular the catalyst, in the reactor may be controlled, by providing water to the process through a plurality of inlets distributed along the periphery of the reactor wall.

Accordingly, the present invention provides a process for preparing ethylene and/or propylene, wherein an oxygenate feedstock is contacted with a molecular sieve-comprising catalyst at a temperature in the range of from 350 to 500° C. to obtain a reactor effluent comprising ethylene and/or propylene and the oxygenate feedstock is contacted with the catalyst in a riser reactor having a reactor wall defining a flow trajectory towards a downstream outlet for reactor effluent, wherein at least oxygenate feedstock and catalyst are provided at one or more upstream inlets of the riser reactor and wherein water quench medium is admitted to the riser reactor at one or more of locations along the length of the flow trajectory through a plurality of inlets distributed along the periphery of the reactor wall.

Reference herein to an oxygenate feedstock is to a feedstock comprising oxygenates.

Reference herein to a water quench medium is to a liquid quench medium comprising water.

Reference herein to water is to water in the liquid phase. Reference herein to steam is to water in the vapour phase.

The conversion of the oxygenate feedstock over a molecular sieve-comprising catalyst to at least ethylene and/or propylene is also referred to as an oxygenate to olefin (OTO) process. Such OTO processes are well known in the art.

The process according to the invention allows for the control of the temperature of the reaction mixture passing through the reactor, and in particular the temperature of the catalyst in the reactor during an oxygenate to olefins process. Where, in prior art processes, heat generated by the exothermic conversion of oxygenates to olefins is removed from the process by providing an aqueous quench medium, such as water, to the reaction mixture through distributor grids extending into the reaction mixture causing premature vaporizing of the water, i.e. inside the distributor grids, the present invention provides the water quench medium through plurality of inlets distributed along the periphery of the reactor wall. As the inlets are provided in the reactor wall, the water does not need to pass through distributor conduits extending into reaction mixture, rather the water quench medium is admitted directly into reaction mixture. The use of a plurality of inlets distributed along the periphery of the reactor wall allows for a good distribution of the water quench medium over the whole of the reactor cross-section. In addition, contrary to distributor grids where high pressure drops over the grid are to be avoided, the present invention, wherein the inlets are located in the reactor wall are less sensitive to high pressure drops and may therefore be designed to handle large pressure drops to induce small liquid droplet formation.

It is an advantage of the process according to the invention that by using water the need to generate steam external to the process is avoided. In order to obtain the same heat consumption, a lower mass flow of water is required compared to steam due to the latent heat of vaporization of water as well as the lower inlet temperature. Therefore, by using water less steam is generated in the process. Due to the risk of hydrothermal deactivation of the catalyst it is preferred to limit the exposure of the catalyst to steam to the extent possible. In addition, less steam needs to be removed from the reactor effluent.

One advantage of the process according to the present invention is that catalyst deactivation due to the exposure of the catalyst to high temperatures may be reduced by controlling the temperature of the reaction mixture in the reactor and maintaining the temperature within an acceptable temperature range. An additional advantage of the process according to the present invention is that operating the process within a narrow temperature range may be beneficial to the selectivity of the process, i.e. result in less coke make and reduced formation of paraffins, compared to a process that is operated over a wide temperature range of the reaction mixture. A narrow temperature range increases the predictability of the process and reduces the risk of hotspots inside the reactor.

In another aspect, the invention provides a reaction system suitable for preparing ethylene and propylene, comprising a riser reactor comprising:

a) an inlet for oxygenate feedstock;
b) an inlet for molecular sieve-comprising catalyst;
c) an outlet for reactor effluent;
d) a reactor wall defining a flow trajectory from the inlets for oxygenate feedstock and zeolite catalyst to the outlet for reactor effluent; and
e) at least one inlet array for providing water quench medium into the reactor, integrated with the reactor wall and
wherein the inlet array comprises a plurality of inlets for water quench medium distributed along the periphery of the reactor wall.

### BRIEF DESCRIPTION OF THE DRAWING

In FIG. 1, a schematic representation is provided of a process according to the invention.

In FIG. 2, an embodiment of a reaction system for preparing ethylene and/or propylene according to the invention is shown.

### DETAILED DESCRIPTION OF THE INVENTION

Ethylene and/or propylene can be produced from oxygenates such as methanol and dimethylether (DME) through an oxygenate-to-olefins (OTO) process. Such processes are well known in the art and are also referred to as methanol-to-olefins or methanol-to-propylene processes. In an OTO process, typically the oxygenate is contacted with a molecular sieve-comprising catalyst at elevated temperatures. In contact with the molecular sieve-comprising catalyst, the oxygenate is converted into at least ethylene and/or propylene. The conversion of the oxygenate into ethylene and propylene is an exothermic process and resultantly a substantial amount of heat of reaction is released during the conversion of the oxygenate. Unless this heat of reaction is withdrawn from the process, it will cause the temperature of the reaction mixture to increase. This temperature increase may have an undesired effect on the catalyst activity. It is known in the art that increased temperatures induce catalyst deactivation. Moreover, it is also believed to be beneficial for the selectivity of the reaction system to operate at near constant temperature. Therefore it is desired to maintain the temperature increase as small as possible.

In the process according to the present invention, oxygenates are converted to at least ethylene and propylene by providing an oxygenate feedstock and molecular sieve-comprising catalyst to a riser reactor and contacting, at elevated temperatures, the oxygenate feedstock and molecular sieve-comprising catalyst in an initial reaction mixture. The initial reaction mixture comprises oxygenate feedstock and molecular sieve-comprising catalyst. Typically, the initial reaction mixture further comprises steam as an inert diluent. As the reaction mixture passes through the riser reactor oxygenate feedstock is consumed while reaction products are formed. Herein both the initial reaction mixture and the mixture formed during the process are referred to as the reaction mixture. As the reaction mixture passes through the riser reactor water quench medium is provided to the reaction mixture. In contact with the reaction mixture, the water evaporates as it heats up, consuming the exothermic heat of reaction released by the oxygenate conversion.

Compared to alternative inert quench media such as paraffins, the heat of evaporation of liquid water and specific heat capacity of the formed steam are high compared on a mass basis. In addition, steam may efficiently be condensed out of the rector effluent, whereas quench media such as paraffins require compression and separation cycles in a separation section. Therefore, water is a particularly suitable quench medium to control the temperature in the riser reactor during the process.

In the process according to the present invention, ethylene and/or propylene are prepared by contacting an oxygenate feedstock with a molecular sieve-comprising catalyst at a temperature in the range of from 350 to 500° C. to obtain a reactor effluent comprising ethylene and/or propylene. The reactor effluent will also comprise the molecular sieve-comprising catalyst and may comprise other hydrocarbons, including oxygenates and C4+ olefins. The reactor effluent will also comprise steam.

In the present invention, the oxygenate feedstock is contacted with the catalyst in a riser reactor. The riser reactor has a reactor wall, which defines a flow trajectory towards a downstream outlet for reactor effluent. Reference herein to the flow trajectory is to a trajectory that the reaction mixture will follow when passed through the riser reactor under normal operation conditions. The riser reactor comprises one or more upstream inlets.

Reference herein to upstream and downstream is to locations along the flow trajectory whereby the flow passes from an upstream location in the direction of a downstream location.

In the process according to the invention, at least oxygenate feedstock and molecular sieve-comprising catalyst are provided at the one or more upstream inlets of the riser reactor and form the reaction mixture. As mentioned above the reaction mixture may comprise diluents such as steam and the reaction mixture, which diluents may be provided at the one or more upstream inlets of the riser reactor.

In the process according to the invention, the water quench medium is admitted to the riser reactor at one or more of locations along the length of the flow trajectory. The water quench medium is admitted to the riser reactor and becomes part of the reaction mixture existing at the location along the length of the flow trajectory where the water quench medium is admitted.

Preferably, these locations where water quench medium is admitted to the riser reactor are located downstream from the one or more upstream inlets of the riser reactor and upstream of the downstream outlet for reactor effluent. Preferably, these locations are separate from the one or more upstream inlets of the riser reactor. For the purposes of this specification, the riser reactor may be defined as a riser reactor comprising two or more serially arranged riser reactor stages, such that the reaction mixture passes from one reactor stage downstream into a subsequent riser reactor stage. The first reactor stage herein comprises the one or more upstream inlets for providing oxygenate feedstock and molecular sieve-comprising catalyst which from the initial reaction mixture, whereas the locations where water quench medium is admitted to the riser reactor are preferably located downstream of the first riser reactor stage in one or more subsequent riser reactor stages. A reactor stage may comprise more than one location where water quench medium is admitted to the riser reactor,

Preferably, the water quench medium is admitted at two or more locations along the flow trajectory. More preferably, the water quench medium is admitted to the riser reactor at in the range of from 2 to 15 locations, even more preferably of from 4 to 10 locations.

The distribution of the water quench medium in the reaction mixture is enhanced by admitted the water quench medium through a plurality of inlets distributed along the cross-sectional periphery of the of the reactor wall. Preferably, the provision of quench medium is evenly distributed over the plurality of inlets. Preferably, at each location along the flow trajectory where water quench medium is admitted, the water quench medium is admitted through in the range of from 5 to 100 inlets, more preferably of from 10 to 100 inlets. By increasing the number of inlets, the even distribution of the water quench medium into the reaction mixture is enhanced. The maximum number of inlets is determined predominantly by design constraint.

The inlets may be integrated in the reactor wall or may be part of an inlet array for providing water quench medium into the reactor, integrated with the reactor wall and comprising a plurality of inlets for water quench medium distributed along the periphery of the reactor wall. Reference herein to inlet array integrated with the reactor wall is to an inlet array that is an integral part of the reactor wall or to an inlet array that is arranged on the inner side of the reactor wall along the periphery of the reactor wall.

It is preferred to evaporate the water quench medium directly subsequent to admitting the water quench medium into the reactor. Fast evaporation allows for a better control and predictability of the temperature of the reaction medium as lag effects caused by water quench medium that is evaporated further downstream of the inlet is reduced.

Preferably, the inlets for admitting the water quench medium are nozzles inducing the formation of a spray. Reference herein to a spray is to flow of small liquid droplets. Preferably, the spray comprises liquid droplets having upon formation an average diameter of in the range of from 0.5 to 1000 μm, preferably of from 1 to 100 μm. A smaller droplet size increases the rate of evaporation due to the increased surfaces area. Spray formation may be enhanced by inducing a pressure drop over the inlet nozzle, preferably the pressure drop over each of the inlet nozzles is in the range of from 2 to 50 bar (gauge), preferably of from 5 to 40 bar (gauge). A preferred means for generating fine spray, with droplet sizes around and preferably below 100 microns, is via twin-fluid atomization, i.e., using liquid pressure of at least 2 bar gauge and gas, such as steam or other process gas. Example of such nozzle device is disclosed in U.S. Pat. No. 5,794,857.

In order to ensure that the water quench medium admitted to the riser reactor is well distributed in the reaction mixture, it is preferred to provide a temporary and localized distortion of the flow through the reactor, in particular upstream to the location where the water quench medium is admitted. The process according to present invention is operated in a riser reactor. The advantage of the use of a riser reactor is that it allows for very accurate control of the contact time of the feed with the catalyst, as riser reactors exhibit a flow of catalyst and reactants through the reactor that approaches plug flow. This type of flow is characterised by a narrow residence time distribution of the catalyst passing through the reactor. Ideally, in a plug flow regime all catalyst particles have the same residence time. However, in reality, friction with the wall of the reactor will result in an accumulation of catalyst at the wall of the reactor, resulting in a broader residence time distribution. The accumulated catalyst, in case of a riser reactor, may flow in an upstream direction along the wall of the reactor, generally referred to as the slip or catalyst slip. In the process according to the invention, it is therefore preferred that the riser reactor comprises one or more obstructing members extending from the inner side of the reactor wall into the flow trajectory. Reference herein to an obstructing member is an object or device that during the process will at least temporarily and localized distort the flow of the reaction mixture. The obstructing member is preferably a ring shaped device, which when placed inside a riser reactor results in a localised decreased inner diameter of the reactor cross-section. The decreased inner diameter being determined by a circular or oval opening in the obstructing member, wherein the central axis of the opening is aligned with the central axis of the riser reactor. Preferably, the obstructing member is a metal or ceramic ring that is attached to the inner side of the reactor wall. Such a ring structure is preferred over prior art obstructing members such as for instance the grid member of U.S. Pat. No. 4,071,573. These grid members are sensitive to blockage and wear by the zeolite comprising catalyst. Where the flow of slipped catalyst encounters the obstruction member according the invention, the direction of the slip is diverted toward the centre of the reactor and into the reaction mixture. The catalyst slip is mixed into the reaction mixture. By diverting the catalyst slip back into the reaction mixture, the obstructing members provide means to narrow the residence time distribution of the catalyst in the riser reactor. It has now been found that this mechanism may be utilised to improve the distribution of the water quench medium admitted to the riser reactor at one or more locations along the flow trajectory. By admitting the water quench medium to the riser reactor downstream of the obstructing members, the diversion of the catalyst slip along the reactor wall into the reaction mixture will improve the distribution of the water quench medium within the reaction mixture and thereby favour a homogenous reduction of the temperature of the reaction mixture. The prior art obstructing members such as for instance the grid member of U.S. Pat. No. 4,071,573 will not, or at least to a much lesser extent, divert the slip flow, and consequently may not enhance the distribution of the water quench medium. The grid like structure affects the flow through the cross-section of the reactor uniformly; whereas the obstructing members according to the present invention only divert the flow near the reactor wall. Preferably, the obstructing member causes the inner diameter to be locally decreased by in the range of from 1 to 25%, preferably of from 2 to 10%, based on the inner diameter of the riser reactor.

When the water quench medium is admitted to the riser reactor downstream of the obstructing members, the temporary distortion of the flow of the reaction mixture will improve the distribution of the water quench medium in the reaction mixture and thereby favour a homogenous reduction of the temperature of the reaction mixture.

The most heat or reaction is released where the oxygenate content in the reaction mixture is the highest, i.e. typically at the upstream end of the flow trajectory.

It has been found that, preferably, the water quench medium is provided to the process at a locations where the oxygenate content in the reaction mixture is high, while as the reaction mixture becomes increasingly depleted in oxygenate less water quench medium needs to be provided. By monitoring the temperature of the reaction mixture at multiple locations along the length of the flow trajectory it is possible to determine whether is required to add further water quench medium.

Preferably, the distance between each subsequent locations along the flow trajectory where quench medium is admitted to the reactor is increased in the downstream direction of the flow trajectory. In providing relatively more locations along the flow trajectory where quench medium is admitted to the reactor at the upstream side of the of the flow trajectory, the water quench medium may be provided where most heat or reaction is released.

Where the reactor wall defines a flow trajectory from the upstream inlets to the downstream outlet for reactor effluent it is preferred that the flow trajectory is defined to have two sections of equal length and more than 50 vol %, more preferably more than 75 vol %, of the total volume of water quench medium admitted to the reactor at any moment is provided to the first, i.e. most upstream, section of the flow trajectory. More preferably, the flow trajectory is defined to have three sections of equal length and more than 50 vol %, more preferably more than 75 vol %, of the total volume of water quench medium admitted to the reactor at any moment is provided to the first, i.e. most upstream, section of the flow trajectory. Even more preferably, the flow trajectory is defined to have four sections of equal length and more than 50 vol %, more preferably more than 75 vol %, of the total volume of water quench medium admitted to the reactor at any moment is provided to the first, i.e. most upstream, section of the flow trajectory. As mentioned above, most of the exothermic reaction heat generated by the process is generated in the initial stages of the process, where the oxygenate content in the reaction mixture is high. By providing the majority of the total volume of water quench medium admitted to the reactor at any moment in the more upstream section of the flow trajectory, the exothermic reaction heat generated by the process may be removed as it is formed, allowing for an optimal control of the temperature of the reaction mixture and reducing exposure of the catalyst to higher than desired temperatures.

It is a particular advantage of the process according to the invention that no heat is withdrawn externally from the process to control the temperature inside the reactor, but rather the temperature controlled by providing a water quench medium inside the reaction mixture. Moreover, as steam is already part of the reaction mixture, at least due to the fact that it is a reaction product, no new components are added to the product slate of the reactor effluent. In addition, the selectivity to ethylene and propylene may be improved with increased steam partial pressure. Admitting water into reactor along the flow trajectory may achieve both the objective of maintaining near iso-thermal condition while at the same time increasing the steam partial pressure thereby improving selectivity to ethylene and propylene.

Preferably, the molecular sieve-comprising catalyst is provided to the riser reactor at a first temperature and reactor effluent, comprising molecular sieve-comprising catalyst, is retrieved from the riser reactor at a second temperature. More preferably, the molecular sieve-comprising catalyst in the reactor effluent has a temperature equal to the second temperature. In the process according to the invention, it is preferred that sufficient water quench medium is admitted to the reactor to maintain a temperature difference between the first and second temperature of in the range of from 0 to 40° C., preferably 0 to 30° C., more preferably 0 to 10° C.

As the reaction mixture passes through reactor along the flow path, the temperature may initially rise as a result of the exothermic reaction taking place in the reaction mixture. Subsequently, the temperature of the reaction mixture decreases as the oxygenate becomes depleted and the endothermic cracking reactions continue.

As a result the axial temperature profile, i.e. in the direction of the flow path, of the reaction mixture may go through a maximum downstream of the one or more upstream inlets. This maximum in axial temperature profile of the reaction mixture is referred to as the maximum temperature, which is the highest temperature to which the reaction mixture, and thus also the catalyst is exposed. Therefore, sufficient water quench medium is admitted to the reactor to maintain a temperature difference between the maximum temperature and the lowest of the first or second temperature of in the range of from 0 to 40° C., preferably 0 to 30° C., more preferably 0 to 10° C.

The exact amount of water quench medium that needs to be admitted to the reactor may depend on many factors. One such factor is the extent of the conversion obtained, which in turn may depend on the catalyst activity and even on the type of zeolite-comprising catalyst used. The desired molar ratio of ethylene and propylene in the reactor effluent will also influence the amount of water quench medium that will need to be admitted. Typically, the choice of catalyst determines the obtained molar ratio of ethylene to propylene. Another factor may be the choice of oxygenate in the oxygenate feedstock. For instance the conversion of DME to olefins is less exothermic than the conversion of methanol to olefins and will therefore require less water quench medium to maintain the temperature of the reaction mixture below desirable levels. Determination of the exact amount of water quench medium that needs to be added and the locations of injection is within the skills of the person skilled in the art based on thermodynamic and reaction kinetic considerations.

Preferably, sufficient water quench medium is admitted along the flow trajectory such that in the range of from 0.0005 to 2.0 mol, preferably of from 0.001 to 1 mol, of water is admitted per mol of oxygenate provided to the process as part of the oxygenate feedstock. Where the oxygenate feedstock provided to the process comprises an ether, such as DME, less water may be required compared to a process where the feed to the process comprises methanol rather than DME. The same applies as the reaction of the ether is less exothermic compared to for instance methanol.

In case of an oxygenate feedstock comprising methanol, preferably, at least 0.1 mol, more preferably 0.2 mol of water per mol of methanol in the oxygenate feedstock are admitted along the flow trajectory.

Steam added as diluent to the initial reaction mixture together with the oxygenate feedstock is considered as part of the water quench medium, as it does not comprise liquid water.

The reaction mixture passes through the riser reactor and exits the reactor as the reactor effluent. The reactor effluent comprises the molecular sieve-comprising catalyst and a gaseous product, comprising ethylene and propylene. The reactor effluent comprises advantageously at least 50 mol %, in particular at least 50 wt %, ethylene and propylene, based on total hydrocarbon content in the reactor effluent.

Typically, the gaseous product also comprises diluents provided to riser reactor together with or as part of the oxygenate feedstock.

In addition to ethylene and/or propylene, the gaseous product may comprise higher olefins, i.e. C4+ olefins, and paraffins. The main by-products from the reaction are C4 and C5 olefins.

Preferably, reactor effluent is subsequently provided to one or more gas/solid separators to retrieve molecular sieve-comprising catalyst from the reactor effluent.

The gas/solid separator may be any separator suitable for separating gases from solids. Preferably, the gas/solid separator comprises one or more centrifugal or cyclone, preferably cyclone, units, optionally combined with a stripper section.

In the gas/solid separator, a gaseous product is separated from the molecular sieve-comprising catalyst. The gaseous product is preferably further treated to retrieve several product fractions from the gaseous product.

The gaseous product will comprise steam. The steam may be separated from the gaseous product and condensed to obtain a water-comprising fraction. The water-comprising faction may be used to prepare at least part of the water quench medium. The water-comprising faction may comprise hydrocarbon components, which are preferably removed prior to using the water-comprising faction as at least part of the water quench medium.

The product fractions will preferably comprise one or more fractions comprising ethylene and/or propylene. The separation of the gaseous product in the mentioned fractions may be done using any suitable work-up section. The design of the work-up section depends on the exact composition of the gaseous product, and may include several separation steps. The design of such a work-up section is well known in the art and does not require further explanation.

Preferably, the product fractions will also comprise one or more fractions comprising C4+ olefins and in particular C4 and/or C5 olefins.

Preferably, where the reactor effluent, and consequently the gaseous product, comprises C4 and/or C5 olefins, the process further comprises subjecting the reaction effluent, and ultimately the gaseous product, to one or more fractionation steps to retrieve at least a fraction comprising C4 and/or C5 olefins from the reaction effluent. Preferably at least part of the fraction comprising C4 and/or C5 olefins is provided to a second reactor, which may be any type of reactor, preferably a riser reactor fluidized bed reactor or fixed bed reactor. In the second reactor, the fraction comprising C4 and/or C5 olefins is, preferably, contacted with a zeolite-comprising catalyst at a temperature in the range of from 500 to 700° C. From the second reactor, a second reactor effluent may be retrieved comprising ethylene and/or propylene. The second reactor effluent comprises advantageously at least 50 mol %, in particular at least 50 wt %, ethylene and propylene, based on total hydrocarbon content in the second reactor effluent.

The conversion of the fraction comprising C4 and/or C5 olefins over a zeolite-comprising catalyst to at least ethylene and/or propylene is also referred to as an olefin cracking process (OCP). Such OCP processes are well known in the art.

The water quench medium comprises water. As mentioned above, water herein refers to liquid water. Preferably, the water quench medium comprises in the range of from 50 to 100 wt % of water, more preferably of from 90 to 100 wt %, even more preferably of from 99.5 to 100 wt % of water, based on the water quench medium. The most preferred water quench medium consists of water. As steam is already part of the reaction mixture, at least due to the fact that it is a reaction product, no new components are added to the product slate of the reactor effluent when water quench medium consists of water. When the water-comprising fraction obtained from the reactor effluent is used as or to prepare the water quench medium it may contain traces, below 200 ppmw based on the total water quench medium, of hydrocarbons and oxygenates, in addition water may comprise dissolved salts, these are not taken into consideration when determine the water content in the water quench medium.

Preferably, the water quench medium admitted has a temperature in the range of from 10 to 99° C., more preferably of from 15 to 50° C.

The oxygenate feedstock provided to the process in the riser reactor comprises oxygenate. The oxygenate used in the oxygenate feedstock provided to the OTO process is preferably an oxygenate which comprises at least one oxygen-bonded alkyl group. The alkyl group preferably is a C1-C5 alkyl group, more preferably C1-C4 alkyl group, i.e. comprises 1 to 5, or 1 to 4 carbon atoms respectively; more preferably the alkyl group comprises 1 or 2 carbon atoms and most preferably one carbon atom. Examples of oxygenates that can be used in the oxygenate feedstock include alcohols and ethers. Examples of preferred oxygenates include alcohols, such as methanol, ethanol, propanol; and dialkyl ethers, such as dimethylether, diethyl ether, methylethyl ether. Preferably, the oxygenate is methanol or dimethylether, or a mixture thereof.

Preferably the oxygenate feedstock comprises at least 50 wt. % of oxygenate, in particular methanol and/or dimethylether, based on total hydrocarbons, i.e. hydrocarbons including oxygenates, more preferably at least 70 wt. %.

In the process the oxygenate feedstock is contacted with the molecular sieve-comprising catalyst.

The oxygenate feedstock is contacted with the catalyst at a temperature in the range of from 350 to 500° C., more preferably of from 375 to 475° C. and a pressure in the range of from 0.1 kPa (1 mbara) to 5 MPa (50 bara), preferably of from 100 kPa (1 bara) to 1.5 MPa (15 bara), more preferably of from 100 kPa (1 bara) to 300 kPa (3 bara). Reference herein to pressures is to absolute pressures.

As mentioned above, the OTO process is operated using a riser reactor. The primary operators for controlling the reaction inside the reactor, and in particular a riser reactor, are the gas residence time, the cat/oil ratio and the feed and catalyst inlet temperature. The gas residence time and the cat/oil ratio may be correlated to the earlier mentioned WHSV.

The gas residence time herein refers to the average time it takes for gas at the reactor, inlet to reach the reactor outlet. The gas residence time is also referred to as τ.

Preferably, the residence time of the reaction mixture in the riser reactor, also referred to as τ, is in the range of from 1 to 10 seconds, more preferably of from 3 to 6 seconds, even more preferably of from 3.5 to 4.5 seconds.

The dimensionless cat/oil ratio herein refers to the mass flow rate of catalyst (kg/h) divided by the mass flow rate of the feed (kg/h), wherein the flow rate of the feed is calculated on a CH₂ basis.

Preferably, the cat/oil ratio i.e. on a CH₂ basis for hydrocarbons including oxygenates, in the riser reactor is in the range of from 1 to 100, more preferably of from of from 1 to 50, even more preferably 5 to 25.

It is preferable to control the severity of the process in the riser reactor. When the process is operated at a too high severity, side reactions increase as well as by-product formation at the cost of ethylene and propylene selectivity. In case, the severity is too low, the process is operated inefficient and sub optimal conversions are obtained. The severity of the process is influenced by several reaction and operation conditions, however a suitable measure for the severity of the process in the riser reactor is the C5 olefin content in the reactor effluent. A higher C5 olefin content indicates lower severity and vice versa. Preferably, the reaction conditions in the riser reactor are chosen such that the reactor effluent comprises in the range of from 2.5 to 40 wt % of C5 olefins, based on the hydrocarbons in the reactor effluent, preferably 4 to 15 wt % of C5 olefins. The C5 content in the reactor effluent is conveniently analyzed using any suitable means of analyzing the hydrocarbon content in a process stream. Particularly suitable means of analyzing the C5 content in the reactor effluent include gas chromatography and near infrared spectroscopy. Preferably, the reaction conditions in the riser reactor are chosen such that the oxygenate conversion is in the range of from 90 to 100%, based on the oxygenates provided to the riser reactor, preferably 95 to 100%.

In addition to the deactivation of the catalyst due to the exposure to high temperature, the catalyst is subject to another, though reversible, deactivation process caused by the deposition of coke on the catalyst in the course of the process. The catalyst may be regenerated by an oxidative regeneration process, whereby at least part of the coke deposits on the catalyst are oxidized

In addition to the oxygenates, also an amount of diluent is provided to the riser reactor together with or as part of the oxygenate feedstock, forming part of the initial reaction mixture.

During the conversion of the oxygenates in the riser reactor, steam is produced as a by-product, which serves as an in-situ produced diluent. Typically, additional steam is added as diluent. The amount of additional diluent that needs to be added depends on the in-situ water make, which in turn depends on the composition of the oxygenate feedstock. Where the diluent provided to the riser reactor together with the oxygenate feedstock is steam, the molar ratio of oxygenate to diluent is between 10:1 and 1:20. Other suitable diluents include inert gases such as nitrogen or methane, but may also include C2-C3 paraffins.

A diluent may also be provided to the second reactor together with the olefins. Preferably, the diluent provided to the second reactor is steam. Other suitable diluents include inert gases such as nitrogen or methane, but may also include C2-C3 paraffins. Preferably, the diluents provided to the first and second reactor are the same, more preferably water or steam.

The molecular sieve-comprising catalyst is a molecular sieve-comprising catalyst suitable for converting the oxygenates to olefins and preferably includes molecular sieve-comprising catalyst compositions. Such molecular sieve-comprising catalyst compositions typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Molecular sieves preferably have a molecular framework of one, preferably two or more corner-sharing [TO4] tetrahedral units, more preferably [SiO4], [AlO4] and/or [PO4] tetrahedral units. These silicon, aluminum and/or phosphorous based molecular sieves and metal containing silicon, aluminum and/or phosphorous based molecular sieves have been described in detail in numerous publications including for example, U.S. Pat. No. 4,567,029. In a preferred embodiment, the molecular sieves have 8-, 10- or 12-ring structures and an average pore size in the range of from about 3 Å to 15 Å.

Preferably, the molecular sieve comprises [PO4] tetrahedral units. Suitable examples of such molecular sieves are silicoaluminophosphates (SAPO), such as SAPO-17, -18, 34, -35, -44, but also SAPO-5, -8, -11, -20, -31, -36, 37, -40, -41, -42, -47 and -56; aluminiophosphates (AlPO) and metal substituted (silico)aluminophosphates (MeAlPO), wherein the Me in MeAlPO refers to a substituted metal atom, including metal selected from one of Group IA, IIA, IB, IIIB, IVB, VB, VIB, VIIB, VIIIB and Lanthanide's of the Periodic Table of Elements, preferably Me is selected from one of the group consisting of Co, Cr, Cu, Fe, Ga, Ge, Mg, Mn, Ni, Sn, Ti, Zn and Zr.

A preferred molecular sieve-comprising catalyst comprises SAPO-34.

In the optional second reactor, the fraction comprising C4 and/or C5 olefins is contacted in an OCP process with a zeolite-comprising catalyst. The fraction comprising C4 and/or C5 olefins provided to the OCP process in the second reactor comprises C4 and/or C5 olefin. The olefin fraction comprising C4 and/or C5 olefins provided to the OCP process is preferably from the gaseous product, which was retrieved from the reactor effluent. Preferably, the fraction comprising C4 and/or C5 olefins includes preferably C4+ olefins, most preferably includes C4 and/or C5 olefins.

Preferably the fraction comprising C4 and/or C5 olefins comprises at least 50 wt. % of olefin, in particular C4 and/or C5 olefin, based on total hydrocarbons, more preferably at least 70 wt. %. In addition to the fraction comprising C4 and/or C5 olefins, other olefin comprising feedstocks may be provided to the second reactor.

The fraction comprising C4 and/or C5 olefins is contacted with the catalyst in the second reactor at a temperature of in the range of from 500 to 700° C., preferably of from 550 to 650° C., more preferably of from 550 to 620° C., even more preferably of from 580 to 610° C.; and a pressure in the range of from 0.1 kPa (1 mbara) to 5 MPa (50 bara), preferably of from 100 kPa (1 bara) to 1.5 MPa (15 bara), more preferably of from 100 kPa (1 bara) to 300 kPa (3 bara). Reference herein to pressures is to absolute pressures. As the cracking of olefins, in particular C5 olefins is an endothermic process it is generally not required to provide measures to reduce the temperature in the second reactor.

The zeolite-comprising catalyst is a zeolite-comprising-comprising catalyst suitable for cracking olefins and preferably includes zeolite-comprising catalyst compositions. Such zeolite-comprising-comprising catalyst compositions typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Zeolites preferably have a molecular framework of one, preferably two or more corner-sharing [TO₄] tetrahedral units, more preferably, two or more [SiO₄], [AlO₄] tetrahedral units.

The zeolite-comprising catalysts include those catalyst containing a zeolite of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48.

Preferred catalysts comprise a more-dimensional zeolite, in particular of the MFI type, more in particular ZSM-5, or of the MEL type, such as zeolite ZSM-11. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. A preferred MFI-type zeolite has a Silica-to-Alumina ratio SAR of at least 60, preferably at least 80.

The zeolite-comprising catalyst may comprise more than one zeolite. In that case it is preferred that the catalyst comprises at least a more-dimensional zeolite, in particular of the MFI type, more in particular ZSM-5, or of the MEL type, such as zeolite ZSM-11, and a one-dimensional zeolite having 10-membered ring channels, such as of the MTT and/or TON type.

The zeolite-comprising catalyst may comprise phosphorus as such or in a compound, i.e. phosphorus other than any phosphorus included in the framework of the zeolite. It is preferred that a catalyst comprising a MEL or MFI-type zeolite additionally comprises phosphorus. The phosphorus may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorus as such or in a compound in an elemental amount of from 0.05 to 10 wt % based on the weight of the formulated catalyst. A particularly preferred catalyst comprises phosphor and MEL or MFI-type zeolites having SAR of in the range of from 60 to 150, more preferably of from 80 to 100. An even more particularly preferred catalyst comprises phosphor and ZSM-5 having SAR of in the range of from 60 to 150, more preferably of from 80 to 100.

It is preferred that zeolites in the hydrogen form are used in the molecular sieve-comprising catalyst, e.g., HZSM-5, HZSM-11, and HZSM-22, HZSM-23. Preferably at least 50 wt %, more preferably at least 90 wt %, still more preferably at least 95 wt % and most preferably 100 wt % of the total amount of zeolite used is in the hydrogen form. It is well known in the art how to produce such zeolites in the hydrogen form.

The catalyst particles, including both the molecular sieve-comprising catalyst as well as the zeolite comprising catalyst used in the process of the present invention can have any shape known to the skilled person to be suitable for this purpose, for it can be present in the form of spray dried catalyst particles. Typically and preferably, Geldart A-class particles are used, see D. Kunii and O. Levenspiel, Fluidization Engineering, 2^{nd} Ed, Butterworth-Heineman, Boston, London, Singapore, Sydney, Toronto, Wellington, 1991, p 77 for Geldart classification of particles. If desired, spent oxygenate conversion catalyst can be regenerated and recycled to the process of the invention. Spray-dried particles allowing use in a fluidized bed or riser reactor system are preferred. Spherical particles are normally obtained by spray drying. Preferably the average particle size is in the range of 1-200 μm, preferably 50-100 μm.

The invention also provides reaction system suitable for preparing ethylene and propylene. The system according to the invention is herein below explained in more detail with reference to the non-limiting Figures.

The person skilled in the art will readily understand that many modifications may be made without departing from the scope of the present invention. Further, the person skilled in the art will readily understand that, while the present invention in some instances may have been illustrated making reference to a specific combination of features and measures, many of those features and measures are functionally independent from others features and measures given in the respective embodiment(s) such that they can be equally or similarly applied independently in other embodiments.

In FIG. 1, a non-limiting schematic representation is provided of a process according to the invention. In the process, a riser reactor 10 is provided, which comprises a riser wall 15. Riser wall 15 defines a flow trajectory 20 through the riser reactor 10.

An oxygenate feedstock 25 is provided to riser reactor 10. In addition, molecular sieve-comprising catalyst 30 is also provided to riser reactor 10. The oxygenate feedstock and molecular sieve-comprising catalyst form a reaction mixture that passes along flow trajectory 20 through the riser reactor at a temperature in the range of from 350 to 500° C. Reactor effluent 35 is retrieved from riser reactor 10 and is passed to gas/solid separator 40.

In gas/solid separator 40, the molecular sieve-comprising catalyst is separated from a gaseous product. The molecular sieve-comprising catalyst 45 is retrieved from the gas/solid separator 40 and may be provided to one or more of a catalyst regeneration facility (not shown), another reactor (not shown) or may be recycled to riser reactor 10 as, or as part of, molecular sieve-comprising catalyst 30. The gaseous product 50 retrieved from gas/solid separator 40 may be provided to a separation section 60. In separation section 60 the gaseous product is treated to condense steam to water and remove the water as water-comprising fraction 62 and to separate the remainder into the desired product fractions. Such treatment may include for instance a water quench to remove steam and one or more compression steps to compress the gaseous product.

At least part of water-comprising fraction 62 may be provided back to riser reactor 10 and admitted to riser reactor 10 at locations 75a, 75b, 75c, and 75d, and optionally further locations (not shown), along flow trajectory 20. Preferably, a part of water-comprising fraction 62 is removed from the process as purge stream 72, to prevent the build-up of undesired hydrocarbon compounds in the process. Optionally, additional external water quench medium 77, preferably pure water, is provided to locations 75a, 75b, 75c, and 75d, and optionally further locations (not shown), through a plurality of inlets distributed along the periphery of the reactor wall 15.

Typically, at least one or more fractions comprising ethylene and propylene 65 are retrieved from separation section 60. However, preferably, also a fraction comprising C4 and/or C5 olefins 70 is retrieved and provided to second riser reactor 80. In second reactor 80, fraction comprising C4 and/or C5 olefins 70 is contacted with molecular sieve-comprising catalyst 90 at a temperature in the range of from 500 to 700° C.

In FIG. 2 is shown an embodiment of a reaction system according to the invention, suitable for preparing ethylene and propylene. Riser reactor 10 comprises an inlet for oxygenate feedstock 225 and an inlet for zeolite catalyst 230. Riser reactor 10 further comprises an outlet for reactor effluent 235.

In riser reactor 10, reactor wall 15 defines a flow trajectory 20 from the inlets 225 to the outlet 235, which passes through the riser reactor.

Riser reactor 10 further comprises at least one inlet array 275 for providing quench medium into the reactor, integrated with the reactor wall 15. Reference herein to inlet array integrated with the reactor wall is to an inlet array that is an integral part of the reactor wall or to an inlet array that is arrange on the inner side of the reactor wall along the periphery of the reactor wall.

The inlet array 275 comprises a plurality of inlets for water quench medium.

Preferably, the inlets for water quench medium are formed by spray nozzles. As described herein above, such nozzles may from a flow of liquid droplets that have a narrow size distribution. In addition such nozzles form small droplets.

Preferably, the inlets for water quench medium are formed by ceramic spray nozzles. Ceramic spray nozzles have the advantage of being wear resistant in contact with the reaction mixture.

In FIG. 2a, a more detailed drawing is provided of a preferred embodiment of an inlet array 275 as may be used in the reactor system according to the invention. The inlet array 275 comprises one or more inlets 300 for water quench medium. The inlet array 275 comprises a plurality of inlets for water quench medium 300 distributed along the periphery 310 of the reactor wall 15. FIG. 2a further shows an obstructing member 320 extending from the reactor wall 15 into the flow trajectory 20.

The obstructing member 320 is preferably a ring shaped device, which when placed inside a riser reactor results in a localised decreased inner diameter of the reactor cross-section. The decreased inner diameter being determined by a circular or oval opening 330 in the obstructing member, wherein the central axis of the opening is aligned along the central axis 340 of the riser reactor. Preferably the obstructing member is a metal or ceramic ring that is attached to the inner side of the reactor wall. Preferably, the obstructing member causes the inner diameter to be locally decreased by in the range of from 1 to 25%, preferably of from 2 to 10%, based on the inner diameter of the riser reactor.

Preferably, the obstruction member is arranged upstream of the plurality of inlets for water quench medium with respect the at least one inlet for oxygenate feed or with respect to the flow trajectory 20.

The invention further provides the use of the reaction system according to the invention in a process according to the invention.

### Examples

The invention is illustrated by the following non-limiting examples.

Tables 1a and 1b and Table 2 show the required amount (mol) of water per mol of methanol converted that needs to be admitted to the process in order to operate the process isothermally, i.e. where zeolite-comprising catalyst is provided to the riser reactor at a first temperature and reactor effluent, comprising zeolite-comprising catalyst, is retrieved from the riser reactor at a second temperature and sufficient water quench medium is admitted to the reactor to maintain a temperature difference between the first and second temperature that is zero. The calculations are based on the heat of formation (ΔH_{f}(T) (kJ/mol)) and ratio of ethylene to propylene in the reactor effluent. It has been assumed that the only reactant is methanol, which is converted as following:

MeOH->H₂O+nC₂H₄=mC₃H₆ (1a)

3m+2n=1 (1b)

(0≦m≦⅓) (1c)

The only products formed are ethylene, propylene and water.

In addition, the heat absorbed as the water heats and evaporates to form steam and the heat absorbed as the steam heats are used calculate the necessary amount water to be admitted.

In Table 1a, calculated values for the heat of formation (□H_{f}(T) (kJ/mol)) of the several reactants and products are shown at different temperatures.

It is assumed that the water is admitted at 25° C. and 1 bar. The water is pressured to 2 bar and heated to its boiling point (120.45° C.). Subsequently, the water is evaporated to give steam at a pressure of 2 bar. The obtained steam is further heated to the reaction temperature. In Table 1b, the calculated enthalpy values water and for steam at several reaction temperatures is provided.

In Table 2, the amount (in mol) of water that needs to be added to attain a temperature increase of the reactor of zero is calculated for two different ethylene to propylene molar ratios in the reactor effluent.

It will be clear from Tables 1a and 1b and Table 2, that the temperature the heat of reaction released by the exothermic conversion of methanol to ethylene and propylene can be absorbed by the evaporation of water admitted to the process to prevent or at least reduce the temperature increase during the process that would have been observed in the absence of the water admission.

Furthermore, it will be clear that the moles of water required to be admitted to the process is essentially independent of the operating temperature.

TABLE 1a

| | |
|---|---|
| ΔH_{f}(T) | T [° C.] |

| [kJ/mol] | 350 | 400 | 450 | 500 |
|---|---|---|---|---|
| MeOH | −211.16 | −212.31 | −213.34 | −214.28 |
| H₂O | −245.07 | −245.50 | −245.92 | −246.32 |
| C2= | 43.90 | 42.92 | 42.03 | 41.22 |
| C3= | 7.62 | 6.18 | 4.88 | 3.72 |

TABLE 1b

| | |
|---|---|
| Enthalpy | Reaction temperature [° C.] |

| | | | | |
|---|---|---|---|---|
| [kJ/mol] | 350 | 400 | 450 | 500 |

| | |
|---|---|
| Water | 1.89 |
| 25° C., 1 bar | |
| Water (boiling) | 9.11 |
| 120.45° C., 2 bar | |
| Steam* | 48.76 |
| 120.45° C., 2 bar | |

| | | | | |
|---|---|---|---|---|
| Steam at the | 57.18 | 59.04 | 60.92 | 62.82 |
| reaction temperature | | | | |
| Heat required** | 55.29 | 57.15 | 59.03 | 60.93 |

*Latent heat of vaporisation 41.22 kJ/mol]
**heat required to convert water of 25° C. and 1 bar to steam at the reaction temperature and 2 bar.

TABLE 2

| | |
|---|---|
| T [K] | Water/MeOH [mol/mol] |

| C₂⁼/C₃⁼ [mol/mol]^{#} | 350 | 400 | 450 | 500 |
|---|---|---|---|---|
| 1:1 | 0.43 | 0.42 | 0.42 | 0.38 |
| 3:7 | 0.49 | 0.49 | 0.48 | 0.44 |

^{#}In the reactor effluent

1. A process for preparing ethylene and/or propylene, wherein an oxygenate feedstock is contacted with a molecular sieve-comprising catalyst at a temperature in the range of from 350 to 500° C. to obtain a reactor effluent comprising ethylene and/or propylene and the oxygenate feedstock is contacted with the catalyst in a riser reactor having a reactor wall defining a flow trajectory towards a downstream outlet for reactor effluent, wherein at least oxygenate feedstock and catalyst are provided at one or more upstream inlets of the riser reactor and wherein a water quench medium is admitted to the riser reactor at one or more of locations along the length of the flow trajectory through a plurality of inlets distributed along the periphery of the reactor wall.

2. A process according to claim 1, wherein the water is admitted to the riser reactor at two or more of locations along the flow trajectory.

3. A process according to claim 1, wherein one or more obstructing members are provided extending from the reactor wall into the flow trajectory and wherein the water quench medium is admitted downstream of the obstructing members, with respect to the flow trajectory.

4. A process according to claim 1, wherein the molecular sieve-comprising catalyst is provided to the riser reactor at a first temperature and reactor effluent, comprising molecular sieve-comprising catalyst, is retrieved from the riser reactor at a second temperature and sufficient water quench medium is admitted to the reactor to maintain a temperature difference between the first and second temperature of in the range of from 0 to 40° C.

5. A process according to claim 1, wherein the reactor effluent comprises C4 and/or C5 olefins and the process further comprises subjecting the reaction effluent to one or more fractionation steps to retrieve at least a fraction comprising C4 and/or C5 olefins from the reaction effluent and providing at least a part of the fraction comprising C4 and/or C5 olefins to a second reactor, contacting the fraction comprising C4 and/or C5 olefins with a zeolite-comprising catalyst at a temperature in the range of from 500 to 700° C. and retrieving from the second reactor a second reactor effluent stream comprising ethylene and/or propylene.

6. A process according to claim 1, wherein the water quench medium consists of water.

7. A process according to claim 1, wherein the molecular sieve-comprising catalyst comprises SAPO-34.

8. A process according to any one of the preceding claims, wherein the oxygenate feedstock comprises methanol and/or dimethylether.

9. A reaction system suitable for preparing ethylene and propylene, comprising a riser reactor comprising:
a) an inlet for oxygenate feedstock;
b) an inlet for molecular-sieve comprising catalyst;
c) an outlet for reactor effluent;
d) a reactor wall defining a flow trajectory from the inlets for oxygenate feedstock and zeolite catalyst to the outlet for reactor effluent; and
e) at least one inlet array for providing water quench medium into the reactor, integrated with the reactor wall and
wherein the inlet array comprises a plurality of inlets for water quench medium distributed along the periphery of the reactor wall.

10. A reaction system according to claim 9, wherein the inlet array comprises an obstructing member extending from the reactor wall into the flow trajectory, preferably wherein the obstruction member is arranged upstream of the inlets for water quench medium, with respect the at least one inlet for oxygenate feedstock.
